(19) United States
(12) Patent Application Publication
(10) Pub. No.: US 2022/0387362 A1
(43) Pub. Date: Dec. 8, 2022

(54) COMPOSITIONS COMPRISING A DHODH INHIBITOR FOR THE TREATMENT OF ACUTE MYELOID LEUKEMIA

(71) Applicant: RHIZEN PHARMACEUTICALS AG, Basel (CH)

(72) Inventors: Srikant VISWANADHA, Hyderabad (IN); Swaroop Kumar Venkata Satya VAKKALANKA, La Chaux de Fonds (CH)

(21) Appl. No.: 17/755,022

(22) PCT Filed: Oct. 20, 2020
(86) PCT No.: PCT/IB2020/059865

§ 371 Date: Apr. 19, 2022

(30) Foreign Application Priority Data
Oct. 21, 2019 (IN) 201941042600

(51) Int. Cl. A61K 31/192; A61K 31/497; A61K 45/06; A61P 35/02

(52) U.S. Cl. CPC A61K 31/192; A61K 31/497; A61K 45/06; A61P 35/02

(57) ABSTRACT

The present invention relates to a method of treatment of acute myeloid leukemia (AML). In one embodiment, the present invention relates to a method of treating acute myeloid leukemia (AML) comprising administering to a subject in need thereof a dihydroorotate dehydrogenase (DHODH) inhibitor, alone in combination with at least one/ms-like tyrosine kinase 3 (FLT-3) inhibitor and/or a DNA polymerase inhibitor.

### PRIORITY DETAILS

The present invention claims the benefit of Indian Provisional Application No. 201941042600, filed 21 Oct. 2019, which is hereby incorporated by reference in its entirety.

### FIELD OF THE INVENTION

The present invention relates to a method of treatment of acute myeloid leukemia (AML). In one embodiment, the present invention relates to a method of treating acute myeloid leukemia (AML) comprising administering to a subject in need thereof a dihydroorotate dehydrogenase (DHODH) inhibitor, alone in combination with at least one fms-like tyrosine kinase 3 (FLT-3) inhibitor and/or a DNA polymerase inhibitor.

### BACKGROUND OF THE INVENTION

Leukemia is a cancerous disease of the bone marrow and the blood. Four types of leukemia can be distinguished: chronic myeloid leukemia, acute myeloid leukemia, chronic lymphoid leukemia and acute lymphoid leukemia.

Myeloid leukamias of the acute type with a rapid progression are called AML or acute myeloid leukemia. Myeloid leukemias of the chronic type with a gradual, less aggressive progression are called CML or chronic myeloid leukemia. These are clonal diseases of the bone marrow characterized by a clonal expansion of myeloid cells which cannot differentiate normally and accumulate in the bone marrow and the blood.

According to the French-American-British (FAB) classification of 1976, there are 8 subtypes of AML, referred to as M0 to M7, depending on the type of cells from which the leukemia develops (Bennett et al., 1976, “Proposals for the classification of the acute leukemias. French-American-British (FAB) co-operative group”. Br. J. Haematol., 33 (4): 451-8).

Recent reports for 2019 estimate around 21,450 people of all ages (11,650 men and boys and 9,800 women and girls) in the United States will at one point be diagnosed with AML

AML has been the second most common type of leukemia diagnosed in adults and children, with most cases occurring in adults. AML makes up 32% of all adult leukemia cases. Around 10,920 deaths (6,290 men and boys and 4,630 women and girls) from AML alone have been estimated to occur in US in 2019.

The 5-year survival rate for people 20 years of age and older with AML has been reported to be ˜24%. For people younger than 20, the survival rate is reported to be around 67%. (see https://www. cancer. net/cancer-types/leukemia-acute-myeloid-aml/statistics).

Recently, much research has been dedicated to the discovery and understanding of the structure and functions of enzymes and bio-molecules associated with various diseases. One such important class of enzymes that has been the subject of extensive research is dihydroorotate dehydrogenase (DHODH).

In the body, DHODH catalyzes the synthesis of pyrimidines, which are necessary for cell growth. Inhibition of DHODH inhibits the growth of (pathologically) fast proliferating cells, whereas cells which grow at normal speed may obtain their required pyrimidine bases from the normal metabolic cycle. The most important types of cells for the immune response, the lymphocytes, exclusively use the synthesis of pyrimidines for their growth and react particularly sensitively to DHODH inhibition.

DHODH inhibition results in decreased cellular levels of ribonucleotide uridine monophosphate (rUMP), thereby arresting proliferating cells in the G1 phase of the cell cycle. The inhibition of de novo pyrimidine nucleotide synthesis is of great interest in view of the observations that lymphocytes seem not to be able to undergo clonal expansion when this pathway is blocked. Substances that inhibit the growth of lymphocytes are important medicaments for the treatment of auto-immune diseases.

During homeostatic proliferation, the salvage pathway which is independent of DHODH seems sufficient for the cellular supply with pyrimidine bases. Only cells with a high turnover and particularly T and B lymphocytes require the de novo pathway to proliferate. In these cells, DHODH inhibition stops the cell cycle progression suppressing DNA synthesis and consequently cell proliferation (see Ann Rheum Dis. 2000 November; 59(11): 841-849).

Therefore, inhibitors of DHODH show beneficial immunosuppressant and antiproliferative effects in human diseases characterized by abnormal and uncontrollable cell proliferation causing chronic inflammation and tissue destruction.

DHODH inhibitors include, for example, leflunomide, teriflunomide, brequinar (NSC 368390) (Cancer Research, 1992, 52, 3521-3527), dichloroallyl lawsone (The Journal of Biological Chemistry, 1986, 261(32), 14891-14895), Maritimus (FK 778) (Drugs of the Future, 2002, 27(8), 733-739), redoxal (The Journal of Biological Chemistry, 2002, 277(44), 41827-41834), DSM265 (Sci. Transl. Med., 2015 Jul. 15; 7(296): 296ra111. doi:10.1126/scitranslmed.aaa6645), BAY2402234 (CAS No. 2225819-06-5, (S)-N-(2-chloro-6-fluorophenyl)-4-(4-ethyl-3-(hydroxymethyl)-5-oxo-4,5-dihydro-1H-1,2,4-triazol-1-yl)-5-fluoro-2-((1,1,1-trifluoropropan-2-yl)oxy)benzamide) (NCT03404726), ASLAN003 (aslanpharma.com/pipeline), PTC299 (DOI: 10.1158/1535-7163.MCT-18-0863 Published January 2019), and BRD9185 (ACS Med. Chem. Lett., 2017, 8, 438-442), ML390 (ACS Med. Chem. Lett., 2016, 7, 12, 1112-1117).

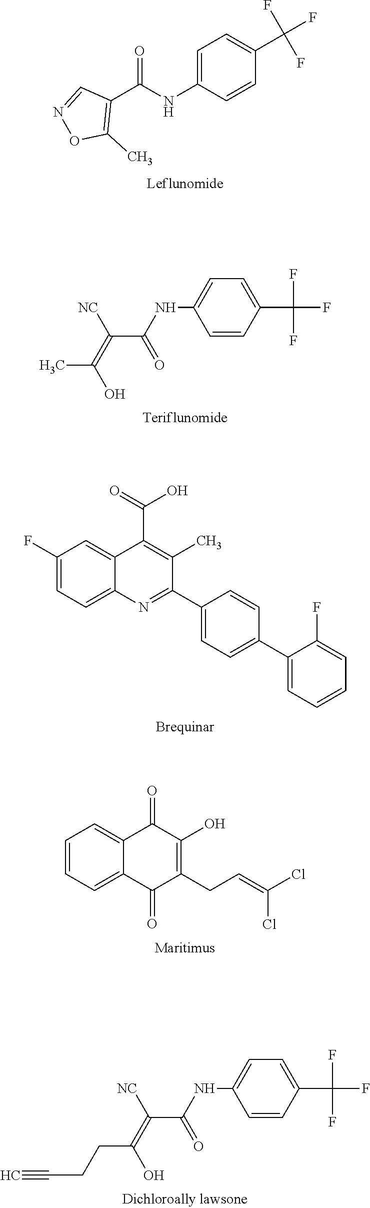 [SMILES: Cc1oncc1C(=O)Nc1ccc(C(F)(F)F)cc1 ; C/C(O)=C(\C#N)C(=O)Nc1ccc(C(F)(F)F)cc1 ; Cc1c(-c2ccc(-c3ccccc3F)cc2)nc2ccc(F)cc2c1C(=O)O ; O=C1C(O)=C(CC=C(Cl)Cl)C(=O)c2ccccc21 ; C#CCC/C(O)=C(\C#N)C(=O)Nc1ccc(C(F)(F)F)cc1]

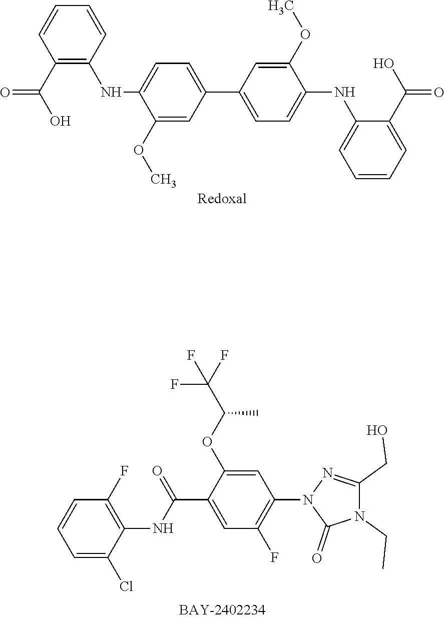 [SMILES: COc1cc(-c2ccc(Nc3ccccc3C(=O)O)c(OC)c2)ccc1Nc1ccccc1C(=O)O ; CCn1c(CO)nn(-c2cc(O[C@@H](C)C(F)(F)F)c(C(=O)Nc3c(F)cccc3Cl)cc2F)c1=O]

In general, inhibitors of DHODH show beneficial immunosuppressive and antiproliferative activities, most pronounced on T-cells (see Fairbanks et al., J. Biol. Chem., 1995, 270, 29682-29689). Leflunomide is used in the treatment of rheumatoid arthritis (see Rozman J. Rheumatol. Suppl., 1998, 53, 27-31;). On the basis of very good efficacy in animal models, brequinar was originally developed for the therapy of organ transplant rejection but was switched to cancer as a secondary indication. The compound failed in the clinic due to its narrow therapeutic window. Oral administration of brequinar and some of its analogues resulted in toxic effects, including leukocytopenia and thrombocytopenia, when given in combination with cyclosporine. See Pally et al., Toxicology, 1998, 127, 207-222. The application of leflunomide might be flawed by its long half-life time of approximately 2 weeks which represents a serious obstacle in patients that have developed side effects (see Fox et al., J. Rheumatol Suppl., 1998, 53, 20-26; Alldred et al., Expert Opin. Pharmacother., 2001, 2, 125-137).

FLT3, a receptor tyrosine kinase (RTK), is a membrane-bound receptor with an intrinsic tyrosine kinase domain FLT3 is composed of an immunoglobulin-like extracellular ligand binding domain, a transmembrane domain, a juxtamembrane dimerization domain and a highly conserved intracellular kinase domain interrupted by a kinase insert. FLT3 belongs to the class III subfamily of RTKs which include structurally similar members such as c-FMS, c-KIT and PDGF receptor. FLT3 is primarily expressed on committed myeloid and lymphoid progenitors with variable expression in the more mature monocytic lineage. FLT3 expression has been described in lymphohematopoietic organs such as the liver, spleen, thymus, and placenta. In the un-stimulated state, FLT3 receptor exists in a monomeric, unphosphorylated form with an inactive kinase moiety. Upon interaction of the receptor with FLT ligand (FL), the receptor undergoes a conformational change, resulting in the unfolding of the receptor and the exposure of the dimerization domain, allowing receptor-receptor dimerization to take place. This receptor dimerization is the prelude to the activation of the tyrosine kinase enzyme, leading to phosphorylation of various sites in the intracellular domain The activated receptor recruits a number of proteins in the cytoplasm to form a complex of protein-protein interactions in the intracellular domain. SHC proteins, GRB2, GRB2-associated binder 2 (GAB2), SHIP, CBL, and CBLB (CBLB related protein) are a few of the many adaptor proteins that interact with the activated FLT3 receptor. As each protein binds to the complex, it becomes activated in turn, resulting in a cascade of phosphorylation reactions that culminates in activation of a number of secondary mediators, including MAP kinase, STAT and AKT/PI3 kinase signal transduction pathways. Once activated, these activated mediators are chaperoned to the nuclear interphase by HSP90, where the message is translocated to the nucleus. In the nucleus, these transcriptional mediators trigger a series of events culminating in regulation of cell differentiation, proliferation apoptosis, and cell survival.

FLT3 activation regulates a number of cellular process (e.g. phospholipid metabolism, transcription, proliferation, and apoptosis), and through these processes, FLT3 activation plays a critical role in governing normal hematopoiesis and cellular growth. Optimum FLT3 function requires the coordinated effort of other growth factors such as SCF, and IL3. Combinations of FL and other growth factors have been found to promote proliferation of primitive hematopoietic progenitor cells as well as more committed early myeloid and lymphoid precursors. FL stimulation appears to mediate differentiation of the early progenitors, where exposure of the hematopoietic progenitors to FL, leads to monocytic differentiation, without significant proliferation. Although FLT3 knockout mice have a subtle phenotype, mice transplanted with FLT3 knock out cells displayed a more global disruption of hamatopoiesis. In addition, if both KIT and FLT3 were knocked out, mice developed severe, life-limiting hematopoietic deficiencies. Thus, the in vitro data and murine knockout models confirm a major role for FLT3 in normal hematopoiesis, especially in times of hematopoietic stress

Expression of FLT3 has been evaluated in hematologic malignancies. The majority of B-cell ALL and AML blasts (>90%) express FLT3 at various levels. Although less frequently and with more variable expression levels, FLT3 receptors are also expressed in other hematopoietic malignancies, including myelodysplasia (MDS), chronic myeloid leukemia (CML), T-cell ALL, and chronic lymphocyctic leukemia (CLL). Data suggest that very high levels of FLT3-WT receptors may promote constitutive activation of the wild-type receptor in malignant cells, and other studies have found that increased FLT3-WT expression in leukemic blasts may be associated with a worse prognosis. (See Soheil Meshinchi et.al., Clin Cancer Res., 2009 Jul. 1; 15(13): 4263-4269)

Acute myeloid leukemia (AML) remains a highly resistant disease to conventional chemotherapy, with a median survival of only 4 months for relapsed and/or refractory disease. Molecular profiling by PCR and next-generation sequencing has revealed a variety of recurrent gene mutations. New agents are rapidly emerging as targeted therapy for high-risk AML. In 1996, FMS-like tyrosine kinase 3/internal tandem duplication (FLT3/ITD) was first recognized as a frequently mutated gene in AML. According to the 2017 ELN risk stratification, patients with FLT3/ITD high-positive AML are classified into adverse risk category. This mutation causes resistance to conventional chemotherapy. Although patients with AML can be cured with hematopoietic stem cell transplantation (HSCT), most of these patients are at high risk for relapse. Thus, the overall cure rate of AML is only 30-40%.

FLT3/ITD gene is found in approximately 30% of patients with AML with normal cytogenetics. FLT3/ITD belongs to the type III family of receptor tyrosine kinases. The FLT3 gene is located on chromosome 13 .q12. It is expressed mainly in human hematopoietic progenitors and dendritic cells and plays key roles in leukemia cell proliferation, differentiation, and survival. Constitutive activation of the FLT3/ITD gene triggers multiple downstream signaling cascades, such as STAT5, RAS, MEK, and PI3K/AKT pathways, and ultimately causes suppression of apoptosis and differentiation of leukemic cells, including dysregulation of leukemic cell proliferation.

Multiple FLT3 inhibitors are in clinical trials for treating patients with FLT3/ITD-mutated AML. (See Mei Wu et.al., Journal of Hematology & Oncology, Vol. 11, Article number: 133 (2018).

Despite currently available intervention therapies, acute myeloid leukemia (AML) remains a significant unmet medical need. Currently, several drugs are available for the treatment of AML and several others under clinical investigation. However, there remains a need for new active therapeutic compounds for the improvement of the strategies for treatment of patients suffering from AML and the development of a treatment alternatives to those already known.

DHODH inhibitors and their preparation are disclosed in International Publication No. WO 11/138665 and in U.S. Pat. No. 8,686,048.

### SUMMARY OF INVENTION

It is an objective of the present invention to provide a method and pharmaceutical compositions for the treatment of acute myeloid leukemia (AML) having a broader therapeutic window over the existing therapies for treating AML, thereby minimizing or obviating possible existing adverse effects generally linked to existing therapies.

Accordingly, in one embodiment, the present invention provides 2-(3′-butoxy-3-chloro-5-fluorobiphenyl-4-ylcarbamoyl)benzoic acid (the compound of formula A shown below) or a pharmaceutically acceptable salt thereof, or a hydrate or solvate thereof, an inhibitor of DHODH, for the treatment of acute myeloid leukemia (AML) as a single agent or in combination with at least one FLT-3 inhibitor and/or DNA polymerase inhibitor.

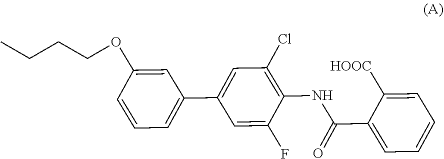 [SMILES: CCCCOc1cccc(-c2cc(F)c(NC(=O)c3ccccc3C(=O)O)c(Cl)c2)c1]

In one aspect, the present invention provides a method of treating acute myeloid leukemia (AML) in a subject in need thereof comprising administering to the subject a dihydroorotate dehydrogenase (DHODH) inhibitor.

In one embodiment, the present invention provides a method of treating acute myeloid leukemia (AML) in a subject in need thereof comprising administering to the subject a dihydroorotate dehydrogenase (DHODH) inhibitor alone or in combination with a FLT-3 inhibitor.

In another embodiment, the present invention provides a method of treating acute myeloid leukemia (AML) in a subject in need thereof comprising administering to the subject a dihydroorotate dehydrogenase (DHODH) inhibitor and at least one FLT-3 inhibitor.

In one embodiment, the present invention provides a method of treating acute myeloid leukemia (AML) in a subject in need thereof, comprising administering to the subject a dihydroorotate dehydrogenase (DHODH) inhibitor alone or in combination with a DNA polymerase inhibitor.

In another embodiment, the present invention provides a method of treating acute myeloid leukemia (AML) in a subject in need thereof comprising administering to the subject a dihydroorotate dehydrogenase (DHODH) inhibitor and at least one DNA polymerase inhibitor.

In one embodiment, the DHODH inhibitor is selected from leflunomide, teriflunomide, brequinar, dichloroallyl lawsone, maritimus (FK 778), redoxal, DSM265, BAY2402234, ASLAN003, PTC299, BRD9185, ML39, a compound of formula (A), and pharmaceutically acceptable salts thereof and hydrates and solvates of any of the foregoing.

In one embodiment of any of the methods, uses or compositions described herein, the dihydroorotate dehydrogenase (DHODH) inhibitor is a compound of formula (A) (shown below) (i.e., 2-(3′-butoxy-3-chloro-5-fluorobiphenyl-4-ylcarbamoyl)benzoic acid) or a pharmaceutically acceptable salt thereof or a hydrate or solvate thereof.

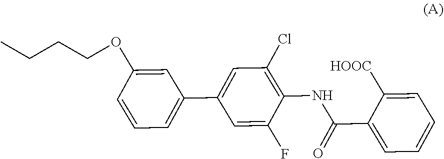 [SMILES: CCCCOc1cccc(-c2cc(F)c(NC(=O)c3ccccc3C(=O)O)c(Cl)c2)c1]

In another embodiment, the present invention provides a method of treating acute myeloid leukemia (AML) in a subject in need thereof comprising administering to the subject (a) BAY2402234 or a pharmaceutically acceptable salt thereof or a hydrate or solvate thereof and (b) at least one FLT-3 inhibitor and/or at least one DNA polymerase inhibitor.

Yet another embodiment is the use of BAY2402234 or a pharmaceutically acceptable salt thereof or a hydrate or solvate thereof in combination with at least one FLT-3 inhibitor and/or a DNA polymerase inhibitor for the treatment of acute myeloid leukemia (AML).

In another embodiment of any of the methods, uses or compositions described herein, the FLT-3 inhibitor is midostaurin, gilteritinib, quizartinib, crenolanib, AKN-028, FF10101, SKLB1028, SKI-G-801, KW-2449, AMG-553, clifutinib, CHMFL-FLT3-335, N-(4-(6-Acetamidopyrimidin-4-yloxy)phenyl)-2-(2-(trifluoromethyl)phenyl) acetamide, SU5614, CG′806 and symadex or a pharmaceutically acceptable salt thereof or a hydrate or solvate thereof.

In one embodiment of any of the methods, uses or compositions described herein, the FLT-3 inhibitor is gilteritinib or a pharmaceutically acceptable salt thereof or a hydrate or solvate thereof.

In another embodiment of any of the methods, uses or compositions described herein, the DNA polymerase inhibitor is cytarabine or a pharmaceutically acceptable salt thereof or a hydrate or solvate thereof.

Another embodiment of the present invention is the use of a compound of formula (A) or a pharmaceutically acceptable salt thereof or a hydrate or solvate thereof for the treatment of AML (e.g., in a subject in need thereof).

Another embodiment of the present invention is the use of a combination of a compound of formula (A) or a pharmaceutically acceptable salt thereof or a hydrate or solvate thereof and at least one FLT-3 inhibitor for the treatment of AML (e.g., in a subject in need thereof).

Another embodiment of the present invention is the use of a combination of a compound of formula (A) or a pharmaceutically acceptable salt thereof or a hydrate or solvate thereof and at least one DNA polymerase inhibitor for the treatment of AML (e.g., in a subject in need thereof).

In one embodiment of any of the methods or uses described herein, the subject is a human.

In one embodiment of any of the methods or uses described herein, a compound of formula (A) or a pharmaceutically acceptable salt thereof or a hydrate or solvate thereof is administered to a subject by the oral route, the intravenous route, the intramuscular route, or the intraperitoneal route. For example, in humans, a preferred administration route is the oral route.

In one embodiment of any of the methods or uses described herein, a compound of formula (A) or a pharmaceutically acceptable salt thereof or a hydrate or solvate thereof and a FLT-3 inhibitor are administered to a subject by the oral route, the intravenous route, the intramuscular route, or the intraperitoneal route. For example, in humans, a preferred administration route is the oral route.

In one embodiment of any of the methods or uses described herein, a compound of formula (A) or a pharmaceutically acceptable salt thereof or a hydrate or solvate thereof and a DNA polymerase inhibitor are administered to a subject by the oral route, the intravenous route, the intramuscular route, or the intraperitoneal route. For example, in humans, a conventional administration route is the oral route.

Another embodiment of the present invention is the use of a compound of formula (A) or a pharmaceutically acceptable salt thereof or a hydrate or solvate thereof, for the preparation of a drug (or medicament) useful for the treatment of AML, where the drug (or medicament) is preferably administered by the oral route.

Another embodiment of the present invention is the use of combination of a compound of formula (A) or a pharmaceutically acceptable salt thereof or a hydrate or solvate thereof, and a FLT-3 inhibitor, for the preparation of a drug (or medicament) useful for the treatment of AML, where the drug (or medicament) is preferably administered by the oral route.

Another embodiment of the present invention is the use of a combination of a compound of formula (A) or a pharmaceutically acceptable salt thereof or a hydrate or solvate thereof, and a DNA polymerase inhibitor, for the preparation of a drug (or medicament) useful for the treatment of AML, where the drug (or medicament) is preferably administered by the oral route.

Yet another embodiment is a method of inhibiting dihydroorotate dehydrogenase in a subject having AML comprising administering to the subject an effective amount of a compound of formula (A) or a pharmaceutically acceptable salt or a hydrate or solvate thereof.

The present invention also provides a method of treating AML in a subject in need thereof comprising administering to the subject a dihydroorotate dehydrogenase inhibitor compound 2-(3′-butoxy-3-chloro-5-fluorobiphenyl-4-ylcarbamoyl)benzoic acid (compound of formula (A)) or a pharmaceutically acceptable salt thereof or a hydrate or solvate thereof.

The present invention also provides a method of treating AML in a subject in need thereof comprising administering to the subject a dihydroorotate dehydrogenase inhibitor compound of formula (A) or a pharmaceutically acceptable salt thereof or a hydrate or solvate thereof and gilteritinib or a pharmaceutically acceptable salt thereof or a hydrate or solvate thereof.

The present invention also provides a method of treating AML in a subject in need thereof comprising administering to the subject a dihydroorotate dehydrogenase inhibitor compound of formula (A) or a pharmaceutically acceptable salt thereof or a hydrate or solvate thereof and cytarabine or a pharmaceutically acceptable salt thereof or a hydrate or solvate thereof.

The present invention further provides a pharmaceutical composition comprising a compound of formula (A) or a pharmaceutically acceptable salt thereof or a hydrate or solvate thereof, and a pharmaceutically acceptable carrier.

The present invention further provides a pharmaceutical composition comprising a compound of formula (A) or a pharmaceutically acceptable salt thereof or a hydrate or solvate thereof, at least one FLT-3 inhibitor, and a pharmaceutically acceptable carrier.

The present invention further provides a pharmaceutical composition comprising a compound of formula (A) or a pharmaceutically acceptable salt thereof or a hydrate or solvate thereof, at least one DNA polymerase inhibitor, and a pharmaceutically acceptable carrier.

The present invention further provides a pharmaceutical composition (e.g., for use in the treatment of acute myeloid leukemia (AML)) comprising (a) BAY2402234 or a pharmaceutically acceptable salt thereof or a hydrate or solvate thereof and (b) at least one FLT-3 inhibitor and/or a DNA polymerase inhibitor, and (c) a pharmaceutically acceptable carrier.

In yet another embodiment, in any of the methods or uses described herein, the DHODH inhibitor alone or in combination with a FLT-3 inhibitor and/or DNA polymerase inhibitor is administered in combination (e.g., administered together or sequentially) with an additional anti-cancer treatment, one or more cytostatic, cytotoxic or anticancer agents, targeted therapy, or any combination of any of the foregoing.

Suitable anti-cancer treatments include, e.g., radiation therapy.

Suitable cytostatic, cytotoxic and anticancer agents include, but are not limited to, DNA interactive agents, such as cisplatin or doxorubicin; topoisomerase II inhibitors, such as etoposide; topoisomerase I inhibitors such as CPT-11 or topotecan; tubulin interacting agents, such as paclitaxel, docetaxel or the epothilones (for example, ixabepilone), either naturally occurring or synthetic; hormonal agents, such as tamoxifen; thymidilate synthase inhibitors, such as 5-fluorouracil; and anti-metabolites, such as methotrexate, other tyrosine kinase inhibitors such as gefitinib (marketed as Iressa®) and erlotinib (also known as OSI-774); angiogenesis inhibitors; EGF inhibitors; VEGF inhibitors; CDK inhibitors; SRC inhibitors; c-Kit inhibitors; Her1/2 inhibitors and monoclonal antibodies directed against growth factor receptors such as erbitux (EGF) and herceptin (Her2), and other protein kinase modulators.

In one embodiment of any of the methods or uses described herein, a compound of formula (A) or a pharmaceutically acceptable salt thereof or a hydrate or solvate thereof are useful for the front-line treatment of acute myeloid leukemia, and for the treatment of relapsed-refractory acute myeloid leukemia.

In one embodiment of any of the methods or uses described herein, a compound of formula (A) or a pharmaceutically acceptable salt thereof or a hydrate or solvate thereof and at least one FLT-3 inhibitor are useful for the front-line treatment of acute myeloid leukemia and for the treatment of relapsed-refractory acute myeloid leukemia.

In one embodiment of any of the methods or uses described herein, a compound of formula (A) or a pharmaceutically acceptable salt thereof or a hydrate or solvate thereof and at least one DNA polymerase inhibitor are useful for the front-line treatment of acute myeloid leukemia and for the treatment of relapsed-refractory acute myeloid leukemia.

In one embodiment, any of the pharmaceutical compositions described herein further comprises one or more cytostatic, cytotoxic or anticancer agents.

In one embodiment, any of the pharmaceutical compositions described herein may be used in combination with one or more anti-cancer treatments, one or more cytostatic, cytotoxic or anticancer agents, targeted therapy, or any combination or any of the foregoing. For example, any of DHODH inhibitors described herein may be used together or sequentially with one or more anti-cancer treatments one or more cytostatic, cytotoxic or anticancer agents, targeted therapy, or any combination or any of the foregoing.

In another embodiment of any of the methods or uses described herein, compound (A) or a pharmaceutically acceptable salt thereof or a hydrate or solvate thereof is administered at a dose of about 5 to about 2000 mg, about 25 to about 1000 mg, about 25 to about 800 mg, about 25 to about 600 mg, about 25 to about 400 mg, or about 25 to about 200 mg.

In another embodiment of any of the methods or uses described herein, the DHODH inhibitor is administered at a dose of i) about 25 to about 1000 mg, ii) about 25 to about 800 mg, iii) about 25 to about 600 mg, iv) about 25 to about 400 mg, or v) about 25 to about 200 mg.

In another embodiment of any of the methods or uses described herein, the DHODH inhibitor is administered at a dose of i) about 50 to 1000 mg, ii) about 50 to about 800 mg, iii) about 50 to about 600 mg, iv) about 50 to about 400 mg, or v) about 50 to about 200 mg.

In another embodiment of any of the methods or uses described herein, the DHODH inhibitor is administered at a dose of i) about 100 to about 1000 mg, ii) about 100 to about 800 mg, iii) about 100 to about 600 mg, iv) about 100 to about 400 mg, or v) about 100 to about 200 mg.

In another embodiment of any of the methods or uses described herein, compound (A) or a pharmaceutically acceptable salt thereof or a hydrate or solvate thereof may be administered as a single dose or in divided doses.

In any of the uses and methods described herein, the subject can be a human subject suffering from relapsed AML, refractory AML, or relapsed-refractory AML.

In any of the uses and methods described herein a compound of formula (A) or a pharmaceutically acceptable salt thereof or a hydrate or solvate thereof is administered orally.

In any of the uses and methods described herein a compound of formula (A) or a pharmaceutically acceptable salt thereof or a hydrate or solvate thereof and at least one FLT-3 inhibitor are administered orally.

In any of the uses and methods described herein a compound of formula (A) or a pharmaceutically acceptable salt thereof or a hydrate or solvate thereof and at least one DNA polymerase inhibitor are administered orally.

Any of the uses and methods described herein allow for treating acute myeloid leukemia with a smaller amount of active compound(s) and/or allow for treating acute myeloid leukemia for a longer period of time.

The pharmaceutical compositions described according to any of the embodiments herein show an activity which is significantly higher than the activity that would have been expected knowing the individual activities of each of the components. Thus, the pharmaceutical compositions described herein allow for treating acute myeloid leukemia with a smaller amount of active compound(s) and/or allow for treating acute myeloid leukemia in a more efficient way.

In any of the methods, use and/or compositions described herein the salt of any of the active compounds described herein may be a salt with pharmacologically acceptable acid or base.

### BRIEF DESCRIPTION OF THE DRAWINGS

FIG. 1 is a bar graph depicting the anti-proliferative effect of compound A in combination with gilteritinib in AML cell line THP-1 according to the procedure described in Example 1B.

FIG. 2 is a bar graph depicting the anti-proliferative effect of compound A in combination with gilteritinib in AML cell line U937 according to the procedure described in Example 1C.

FIG. 3 is a bar graph depicting the effect of compound A on CD11b mRNA expression in THP-1 cell lines according to the procedure described in Example 2.

FIG. 4 is a bar graph depicting the effect of compound A on CD11b expression in THP-1 cell lines according to the procedure described in Example 2A.

FIG. 5 is a bar graph depicting the effect of compound A on CD11b expression in MV411 cell lines according to the procedure described in Example 2A.

FIG. 6 is a bar graph depicting the effect of compound A in combination with gilteritinib on p-Akt expression in THP-1 cell lines according to the procedure described in Example 3.

FIG. 7 is a bar graph depicting the effect of compound A in combination with gilteritinib on p-Erk 1/2 expression in THP-1 cell lines according to the procedure described in Example 3.

FIG. 8 is a bar graph depicting the effect of compound A in combination with cytarabine on tumor weight in MV411 xenograft model according to the procedure described in Example 4.

FIG. 9 is a line graph depicting the effect of compound A in combination with cytarabine on tumor volume in MV411 xenograft model according to the procedure described in Example 4.

### DETAILED DESCRIPTION OF THE INVENTION

### Definitions

Unless defined otherwise, all technical and scientific terms used herein have the same meaning as is commonly understood in the field to which the subject matter belongs. In the event that there is a plurality of definitions for terms herein, those in this section prevail. Where reference is made to a URL or other such identifier or address, it is understood that such identifiers generally change and particular information on the internet comes and goes, but equivalent information is found by searching the internet. Reference thereto evidences the availability and public dissemination of such information.

It is to be understood that the foregoing general description and the following detailed description are exemplary and explanatory only and are not restrictive of any subject matter.

In this application, the use of the singular includes the plural unless specifically stated otherwise. It must be noted that, as used in the specification, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

Definition of standard chemistry and molecular biology terms may be found in reference works including, but not limited to, Carey and Sundberg “ADVANCED ORGANIC CHEMISTRY 4th edition” Vols. A (2000) and B (2001), Plenum Press, New York and “MOLECULAR BIOLOGY OF THE CELL 5th edition” (2007), Garland Science, New York. Unless otherwise indicated, conventional methods of mass spectroscopy, NMR, HPLC, protein chemistry, biochemistry, recombinant DNA techniques and pharmacology are contemplated within the scope of the embodiments disclosed herein.

Unless specific definitions are provided, the nomenclature employed in connection with, and the laboratory procedures and techniques of, analytical chemistry, and medicinal and pharmaceutical chemistry described herein are those generally used. In some embodiments, standard techniques are used for chemical analyses, pharmaceutical preparation, formulation, and delivery, and treatment of patients. In other embodiments, standard techniques are used for recombinant DNA, oligonucleotide synthesis, and tissue culture and transformation (e.g., electroporation, lipofection). In certain embodiments, reactions and purification techniques are performed e.g., using kits of manufacturer's specifications or as described herein. The foregoing techniques and procedures are generally performed of conventional methods and as described in various general and more specific references that are cited and discussed throughout the present specification.

Pharmaceutically acceptable salts forming part of this invention include salts derived from inorganic bases such as Li, Na, K, Ca, Mg, Fe, Cu, Zn, and Mn; salts of organic bases such as N,N′-diacetylethylenediamine, glucamine, triethylamine, choline, hydroxide, dicyclohexylamine, metformin, benzylamine, trialkylamine, and thiamine; salts of chiral bases such as alkylphenylamine, glycinol, and phenyl glycinol; salts of natural amino acids such as glycine, alanine, valine, leucine, isoleucine, norleucine, tyrosine, cystine, cysteine, methionine, proline, hydroxy proline, histidine, omithine, lysine, arginine, and serine; quaternary ammonium salts of the compounds of invention with alkyl halides, alkyl sulphates such as Mel and (Me)2SO4; salts of non-natural amino acids such as D-isomers or substituted amino acids; salts of guanidine; and salts of substituted guanidine wherein the substituents are selected from nitro, amino, alkyl, alkenyl, alkynyl, ammonium or substituted ammonium salts and aluminum salts. Salts may include acid addition salts where appropriate which are sulphates, nitrates, phosphates, perchlorates, borates, hydrohalides, acetates, tartrates, maleates, citrates, fumarates, succinates, palmoates, methanesulphonates, benzoates, salicylates, benzenesulfonates, ascorbates, glycerophosphates, and ketoglutarates.

When ranges are used herein for physical properties, such as molecular weight, or chemical properties, all combinations and sub-combinations of ranges and specific embodiments therein are intended to be included. The term “about” when referring to a number or a numerical range means that the number or numerical range referred to is an approximation within experimental variability (or within statistical experimental error), and thus the number or numerical range may vary from, for example, between 1% and 15% of the stated number or numerical range. The term “comprising” (and related terms such as “comprise” or “comprises” or “having” or “including”) includes those embodiments, for example, an embodiment of any composition of matter, composition, method, or process, or the like, that “consist of” or “consist essentially of” the described features.

Abbreviations used herein have their conventional meaning within the chemical and biological arts, unless otherwise indicated.

The term “effective amount” or “therapeutically effective amount” refers to that amount of a compound described herein that is sufficient to effect the intended application including, but not limited to, disease treatment, as defined below. The therapeutically effective amount may vary depending upon the intended application (in vitro or in vivo), or the subject and disease condition being treated, e.g., the weight and age of the subject, the severity of the disease condition, the manner of administration and the like, which can readily be determined by one of ordinary skill in the art. The term also applies to a dose that will induce a particular response in target cells, e.g., reduction of platelet adhesion and/or cell migration. The specific dose will vary depending on the particular compounds chosen, the dosing regimen to be followed, whether it is administered in combination with other compounds, timing of administration, the tissue to which it is administered, and the physical delivery system in which it is carried.

As used herein, the terms “treatment” and “treating” refer to an approach for obtaining beneficial or desired results including, but not limited to, therapeutic benefit and/or a prophylactic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient may still be afflicted with the underlying disorder. For prophylactic benefit, the compositions may be administered to a patient at risk of developing a particular disease, or to a patient reporting one or more of the physiological symptoms of a disease, even though a diagnosis of this disease may not have been made.

The term “front-line treatment” refers to the first treatment given for a disease. It is often part of a standard set of treatments, such as surgery followed by chemotherapy and radiation. When used by itself, front-line therapy is generally the one accepted as the best treatment. If it does not cure the disease or it causes severe side effects, other treatment may be added or used instead. It is also called induction therapy, primary therapy, and primary treatment.

The term “relapsed” refers to disease that reappears or grows again after a period of remission.

The term “refractory” is used to describe when the cancer does not respond to treatment (meaning that the cancer cells continue to grow) or when the response to treatment does not last very long.

The term “subject” or “patient” refers to an animal, such as a mammal, for example a human The methods described herein can be useful in both human therapeutics and veterinary applications. In some embodiments, the patient is a mammal, and in some embodiments, the patient is human For veterinary purposes, the terms “subject” and “patient” include, but are not limited to, farm animals including cows, sheep, pigs, horses, and goats; companion animals such as dogs and cats; exotic and/or zoo animals; laboratory animals including mice, rats, rabbits, guinea pigs, and hamsters; and poultry such as chickens, turkeys, ducks, and geese.

The term “carrier,” as used herein, refers to relatively nontoxic chemical compounds or agents that facilitate the incorporation of an active compound into cells or tissues.

The terms “pharmaceutically acceptable carrier” and “pharmaceutically acceptable excipient” include, but are not limited to, any and all solvents, dispersion media, coatings, antibacterial and antifungal agents, isotonic and absorption delaying agents, one or more suitable diluents, fillers, salts, disintegrants, binders, lubricants, glidants, wetting agents, controlled release matrices, colorants, flavorings, carriers, excipients, buffers, stabilizers, solubilizers, and any combination of any of the foregoing. Except insofar as any conventional media or agent is incompatible with the active ingredient(s), its use in the therapeutic compositions of the invention is contemplated. Supplementary active ingredients can also be incorporated into the compositions.

The term “diluent” refers to chemical compounds that are used to dilute the compound of interest prior to delivery. In some embodiments, diluents are used to stabilize compounds because they provide a more stable environment. Salts dissolved in buffered solutions (which also provide pH control or maintenance) are utilized as diluents, including, but not limited to a phosphate buffered saline solution.

The term “glidant” is a substance that use to increase the flowability of a powder. That means it promotes the flow of the tablet granules (or the powder). It does so by reducing the friction between these granules. Suitable glidants include, but are not limited to, fumed silicon dioxide, sodium aluminosilicate, calcium silicate, powdered cellulose, colloidal silicon dioxide, microcrystalline cellulose, corn starch, sodium benzoate, calcium carbonate, magnesium carbonate, talc, metallic stearates, calcium stearate, magnesium stearate, zinc stearate, magnesium lauryl sulfate, and magnesium oxide, or a mixture thereof.

The term “filler” refers to a substance that adds bulk to products making very small active ingredient components easy for consumer to take. Suitable fillers include, but are not limited to, calcium carbonate, dibasic calcium phosphate, lactose, magnesium carbonate, magnesium oxide, lactose anhydrous, microcrystalline cellulose, insomalt, mannitol and any mixtures thereof, more preferably isomalt and/or microcrystalline cellulose.

The term “lubricant” refers to a substance that is used to prevent the clumping of active ingredients and prevent the sticking of materials to machines in the manufacturing plant. Suitable lubricants include, but are not limited to, stearic acid, a salt of stearic acid, talc, sodium stearyl fumarate, calcium stearate, glyceryl behenate, magnesium silicate, magnesium trisilicate, hydrogenated castor oil or mixtures thereof.

The terms “disintegrant” and “disintegrator” refer to a substance that allows for breakdown of a capsule or tablet when wet. This ensures rapid breakdown to facilitate rapid absorption of a product. Suitable disintegrants include, but are not limited to, sodium starch glycolate, starch, croscarmellose sodium, crospovidone, carboxymethyl cellulose calcium, carboxymethylcellulose sodium, magnesium aluminium silicate or mixtures thereof.

The term “binder” refers to a substance that is used to hold ingredients together. They also give weight and allow small active ingredients to be combined into an easy to take capsule or tablet. Binders are typically sugar derivatives. Suitable binders include, but are not limited to, hydroxypropyl cellulose, polyvinylpyrrolidone k-30, hydroxypropyl cellulose (low-substituted), starch or mixtures thereof, more preferably hydroxypropyl cellulose (low-substituted).

The term “co-administration,” “administered in combination with,” and their grammatical equivalents, as used herein, encompasses administration of two or more agents to a subject so that both agents and/or their metabolites are present in the animal at the same time. Co-administration includes simultaneous administration in separate compositions, administration at different times in separate compositions, or administration in a composition in which both agents are present.

### METHODS OF TREATMENT AND USES

In any of the methods of treatment and uses described herein, one or more additional active agents can be administered with the compound of formula (A) or a pharmaceutically acceptable salt thereof or a hydrate or solvate thereof. For example, the compound of formula (A) or a pharmaceutically acceptable salt thereof or a hydrate or solvate thereof may be administered in combination (administered together or sequentially) with one or more known anti-cancer treatments such as chemotherapy, radiation therapy, biological therapy, bone marrow transplantation, stem cell transplant or any other anticancer therapy or with one or more cytostatic, cytotoxic or anticancer agents or targeted therapy either alone or in combination, such as but not limited to, for example, DNA interactive agents, such as fludarabine, cisplatin, chlorambucil, bendamustine or doxorubicin; alkylating agents, such as cyclophosphamide; topoisomerase II inhibitors, such as etoposide; topoisomerase I inhibitors such as CPT-11 or topotecan; tubulin interacting agents, such as paclitaxel, docetaxel or the epothilones (for example ixabepilone), either naturally occurring or synthetic; hormonal agents, such as tamoxifen; thymidilate synthase inhibitors, such as 5-fluorouracil; and anti-metabolites, such as methotrexate; other tyrosine kinase inhibitors such as gefitinib (Iressa®) and OSI-774; angiogenesis inhibitors; EGF inhibitors; VEGF inhibitors; CDK inhibitors; SRC inhibitors; c-Kit inhibitors; Her1/2 inhibitors, checkpoint kinase inhibitors and monoclonal antibodies directed against growth factor receptors such as erbitux (EGF) and herceptin (Her2); CD20 monoclonal antibodies such as rituximab, ublixtumab (TGR-1101), ofatumumab (HuMax; Intracel), ocrelizumab, veltuzumab, GA101(obinutuzumab), AME-133v (LY2469298, Applied Molecular Evolution), ocaratuzumab (Mentrik Biotech), PRO131921, tositumomab, hA20 (Immunomedics, Inc.), ibritumomab-tiuxetan, BLX-301 (Biolex Therapeutics), rituximab (Reditux®) (Dr. Reddy's Laboratories), and PRO70769 (described in WO2004/056312); other B-cell targeting monoclonal antibodies such as belimumab, atacicept or fusion proteins such as blisibimod and BR3-Fc, other monoclonal antibodies such as alemtuzumab and other protein kinase modulators.

The methods of treatment and uses described herein also include use of one or more additional active agents (or a regimen of one or more additional active agents) to be administered with the compound of formula (A) or a pharmaceutically acceptable salt thereof or a hydrate or solvate thereof. For example CHOP (cyclophosphamide, doxorubicin, vincristine, prednisone); R-CHOP (rituximab-CHOP); hyperCV AD (hyperfractionated cyclophosphamide, vincristine, doxorubicin, dexamethasone, methotrexate, cytarabine); R-hyperCV AD (rituximab-hyperCV AD); FCM (fludarabine, cyclophosphamide, mitoxantrone); R-FCM (rituximab, fludarabine, cyclophosphamide, mitoxantrone); bortezomib and rituximab; temsirolimus and rituximab; temsirolimus and Velcade®; Iodine-131 tositumomab (Bexxar®) and CHOP; CVP (cyclophosphamide, vincristine, prednisone); R-CVP (rituximab-CVP); ICE (iphosphamide, carboplatin, etoposide); R-ICE (rituximab-ICE); FCR (fludarabine, cyclophosphamide, rituximab); FR (fludarabine, rituximab); and D.T. PACE (Dexamethasone, Thalidomide, Cisplatin, Adriamycin, Cyclophosphamide, Etoposide).

The DHODH compounds described herein are also useful in combination (administered together or sequentially) with one or more steroidal anti-inflammatory drugs, non-steroidal anti-inflammatory drugs (NSAIDs) or immune selective anti-inflammatory Derivatives (ImSAIDs).

According to one embodiment, the compound of formula (A) or a hydrate, a pharmaceutically acceptable salt or a solvate thereof can also be administered in combination with one or more other active principles useful in one of the pathologies mentioned above, for example an anti-emetic, analgesic, anti-inflammatory or anti-cachexia agent.

It is also possible to combine any of the methods, uses and/or compounds described herein with a radiation treatment.

It is also possible to combine any of the methods, uses and/or compounds described herein with surgery including either pre, post, or during period of surgery.

These treatments can be administered simultaneously, separately, sequentially and/or spaced in time.

### PHARMACEUTICAL COMPOSITIONS

Any of the pharmaceutical compositions described herein may comprise a DHODH inhibitor (such as Compound (A) or a pharmaceutically acceptable salt thereof or a hydrate or solvate thereof) and optionally one or more pharmaceutically acceptable carriers or excipients.

In one embodiment, the pharmaceutical compositions described herein may comprise a DHODH inhibitor or a pharmaceutically acceptable salt thereof or a hydrate or solvate thereof and at least one FLT-3 inhibitor and optionally one or more pharmaceutically acceptable carriers or excipients.

In another embodiment, the pharmaceutical compositions described herein may comprise a DHODH inhibitor or a pharmaceutically acceptable salt thereof or a hydrate or solvate thereof and at least one DNA polymerase inhibitor and optionally one or more pharmaceutically acceptable carriers or excipients.

In one embodiment, the pharmaceutical composition comprises a therapeutically effective amount of a DHODH inhibitor, such as Compound (A) or a hydrate, pharmaceutically acceptable salt or solvate thereof. The pharmaceutical composition may include one or more additional active ingredients, as described according to any embodiment herein.

In another embodiment, the pharmaceutical composition comprises a therapeutically effective amount of a DHODH inhibitor, such as compound (A) or a pharmaceutically acceptable salt thereof or a hydrate or solvate thereof, and an FLT-3 inhibitor such as gilteritinib. The pharmaceutical composition may include one or more additional active ingredients, as described herein.

In another embodiment, the pharmaceutical composition includes a therapeutically effective amount of a DHODH inhibitor, such as compound (A) or a pharmaceutically acceptable salt thereof or a hydrate or solvate thereof, and a DNA polymerase inhibitor such as cytarabine or a pharmaceutically acceptable salt thereof or a hydrate or solvate thereof. The pharmaceutical composition may include one or more additional active ingredients, as described herein.

Suitable pharmaceutical carriers and excipients may be selected from diluents, fillers, salts, disintegrants, binders, lubricants, glidants, wetting agents, controlled release matrices, colorants, flavorings, buffers, stabilizers, solubilizers, and any combination of any of the foregoing.

The pharmaceutical compositions described herein can be administered alone or in combination with one or more other active agents. Where desired, the DHODH inhibitor and other agent(s) may be mixed into a preparation or both components may be formulated into separate preparations to use them in combination separately or at the same time.

The pharmaceutical compositions described herein can be administered alone or in combination with one or more other active agents. Where desired, the DHODH inhibitor and FLT-3 inhibitor and optionally other agent(s) may be mixed into a preparation or both components may be formulated into separate preparations to use them in combination separately or at the same time.

The pharmaceutical compositions described herein can be administered alone or in combination with one or more other active agents. Where desired, the DHODH inhibitor and DNA polymerase inhibitor and optionally other agent(s) may be mixed into a preparation or both components may be formulated into separate preparations to use them in combination separately or at the same time.

The pharmaceutical compositions described herein can be administered together or in a sequential manner with one or more other active agents. Where desired, the DHODH inhibitor and other agent(s) may be co-administered or both components may be administered in a sequence to use them as a combination.

The pharmaceutical compositions described herein can be administered together or in a sequential manner with one or more other active agents. Where desired, the DHODH inhibitor and FLT-3 inhibitor and other agent(s) may be co-administered, or all the components may be administered in a sequence to use them as a combination.

The pharmaceutical compositions described herein can be administered together or in a sequential manner with one or more other active agents. Where desired, the DHODH inhibitor and DNA polymerase inhibitor and optionally other agent(s) may be co-administered or all the components may be administered in a sequence to use them as a combination.

The DHODH inhibitor alone or in combination with FLT-3 inhibitor and/or DNA polymerase inhibitor and its pharmaceutical compositions described herein can be administered by any route that enables delivery of the DHODH inhibitor to the site of action, such as orally, intranasally, topically (e.g., transdermally), intraduodenally, parenterally (including intravenously, intraarterially, intramuscularally, intravascularally, intraperitoneally or by injection or infusion), intradermally, by intramammary, intrathecally, intraocularly, retrobulbarly, intrapulmonary (e.g., aerosolized drugs) or subcutaneously (including depot administration for long term release e.g., embedded-under the-splenic capsule, brain, or in the cornea), sublingually, anally, rectally, vaginally, or by surgical implantation (e.g., embedded under the splenic capsule, brain, or in the cornea).

The pharmaceutical compositions described herein can be administered in solid, semi-solid, liquid or gaseous form, or may be in dried powder, such as lyophilized form. The pharmaceutical composition can be packaged in forms convenient for delivery, including, for example, solid dosage forms such as capsules, sachets, cachets, gelatins, papers, tablets, suppositories, pellets, pills, troches, and lozenges. The type of packaging will generally depend on the desired route of administration. Implantable sustained release formulations are also contemplated, as are transdermal formulations.

The pharmaceutical composition may, for example, be in a form suitable for oral administration as a tablet, capsule, pill, powder, sustained release formulations, solution, suspension, for parenteral injection as a sterile solution, suspension or emulsion, for topical administration as an ointment or cream or for rectal administration as a suppository. The pharmaceutical composition may be in unit dosage forms suitable for single administration of precise dosages.

Oral solid dosage forms are described in, e.g., Remington's Pharmaceutical Sciences, 20th Ed., Lippincott Williams & Wilkins., 2000, Chapter 89, “Solid dosage forms include tablets, capsules, pills, troches or lozenges, and cachets or pellets”. Also, liposomal or proteinoid encapsulation may be used to formulate the compositions (as, for example, proteinoid microspheres reported in U.S. Pat. No. 4,925,673). Liposomal encapsulation may include liposomes that are derivatized with various polymers (e.g., U.S. Pat. No. 5,013,556).

The pharmaceutical compositions described herein may include a DHODH inhibitor and inert ingredients which protect against degradation in the stomach and which permit release of the biologically active material in the intestine.

The amount of the DHODH inhibitor, such as Compound (A) or a pharmaceutically acceptable salt thereof or a hydrate or solvate thereof to be administered is dependent on the mammal being treated, the severity of the disorder or condition, the rate of administration, the disposition of the compound and the discretion of the prescribing physician. In certain embodiments, an effective dosage is in the range of about 0.001 to about 100 mg per kg body weight per day, preferably about 1 to about 35 mg/kg/day, in single or divided doses. For a 70 kg human, this would amount to about 0.05 to about 7 g/day, preferably about 0.05 to about 2.5 g/day. An effective amount of a DHODH inhibitor, such as compound (A) or a pharmaceutically acceptable salt thereof or a hydrate or solvate thereof of the invention may be administered in either single or multiple doses (e.g., two or three times a day).

The amount of the FLT-3 inhibitor, such as gilteritinib or a pharmaceutically acceptable salt thereof or a hydrate or solvate thereof, or the amount of the DNA polymerase inhibitor, such as cytarabine or a pharmaceutically acceptable salt thereof or a hydrate or solvate thereof to be administered is dependent on the mammal being treated, the severity of the disorder or condition, the rate of administration, the disposition of the compound and the discretion of the prescribing physician. However, an effective dosage for each inhibitor may be in the range of about 0.001 to about 100 mg per kg body weight per day, preferably about 1 to about 35 mg/kg/day, in single or divided doses. For a 70 kg human, this would amount to about 0.05 to about 7 g/day, preferably about 0.05 to about 2.5 g/day. An effective amount of the FLT-3 inhibitor and/or the DNA polymerase inhibitor may be administered in either single or multiple doses (e.g., two or three times a day).

More preferably, in any of the methods and uses described herein, the DHODH inhibitor is a compound of Formula (A) or a pharmaceutically acceptable salt thereof or a hydrate or solvate thereof.

A further embodiment of the present invention relates to a method of treating AML comprising administering a therapeutically effective amount of a pharmaceutical composition according to any of the embodiments described herein to a subject in need thereof (preferably, a human subject) in need thereof.

A further embodiment of the present invention relates to the use of a pharmaceutical composition according to any of the embodiments described herein in the preparation of a medicament for treating haematological and solid cancers, e.g., AML.

The following general methodology described herein provides the manner and process of using the DHODH inhibitor alone or in combination with FLT-3 inhibitor and/or DNA polymerase inhibitor and are illustrative rather than limiting. Further modification of provided methodology and additionally new methods may also be devised in order to achieve and serve the purpose of the invention. Accordingly, it should be understood that there may be other embodiments which fall within the spirit and scope of the invention as defined by the specification hereto

### Preparation of Compound A

### Intermediate 1: 3′-butoxy-3-chloro-5-fluorobiphenyl-4-amine

The title compound (3′-butoxy-3-chloro-5-fluorobiphenyl-4-amine) (0.190 g) was prepared from 4-bromo-2-chloro-6-fluoroaniline (0.2 g, 0.89 mmol) and 3-butoxyphenylboronic acid (0.224 g, 1.16 mmol) by using a Suzuki coupling reaction in the presence of tetrakis(triphenylphosphine)palladium(0) (0.08 eq.) and potassium carbonate (3.3 eq.). The mixture was degassed with N₂ for 30 min. and refluxed until both the starting materials disappeared as monitored by TLC. Work-up (H₂O/AcOEt) and purification gave the desired product as a yellow solid (0.19 g). ¹H-NMR (δ ppm, DMSO-d₆, 400 MHz): 7.44-7.41 (m, 2H), 7.27 (t, J 7.9, 1H), 7.17-7.10 (m, 2H), 6.81-6.84 (m, 1H), 5.50 (s, 2H), 4.01 (t, J 5.3, 2H), 1.72-1.65 (m, 2H), 1.50-1.41 (m, 2H), 0.93 (t, J 7.4, 3H).

### Compound A: 2-(3′-butoxy-3-chloro-5-fluorobiphenyl-4-ylcarbamoyl)benzoic acid

Intermediate 1 (90 mg, 0.31 mmol) was dissolved in ˜2 ml of acetic acid Phthalic anhydride (90 mg, 0.6 mmol) was added to the mixture and stirred at room temperature overnight. The solid that separated out was filtered and washed with petroleum ether and dried under vacuum to obtain the title compound (39 mg) as a white solid. M.P.: 128-130° C. ¹H-NMR (δ ppm, DMSO-d₆, 400 MHz): 13.02 (s, 1H), 10.23 (s, 1H), 7.82 (d, J 7.9, 1H), 7.73 (s, 1H), 7.60-7.57 (m, 4H), 7.37 (t, J 7.9, 1H), 7.32-7.25 (m, 2H), 6.99-6.96 (m, 1H), 4.06 (t, J 6.4, 2H), 1.73-1.68 (m, 2H), 1.45 (h, J 7.5, 2H), 0.94 (t, J 7.4, 3H). MS (m/z): 440.19 ([M-H]⁻).

The present invention is now further illustrated by means of the following biological examples.

### BIOLOGICAL EXAMPLES

Provided below are illustrative examples of the use of a DHODH inhibitor alone or in combination with a FLT-3 inhibitor or a DNA polymerase inhibitor which provides and establishes a synergic effect for the combination when compared to the effect of the individual DHODH inhibitor or the FLT-3 inhibitor or DNA polymerase inhibitor alone.

### Example 1

### Anti-Proliferative Effect of Compound A in AML Cell Lines (MTT Assay)

Compound A was tested across a panel of AML cell lines (U937, HL-60, THP-1, KG-1 and MV411). Cells were plated in 96-well plates and incubated with desired concentrations of Compound A for 72 hours (h). At the end of the incubation period, MTT ((3-(4,5-dimethylthiazol-2-yl)-2,5-diphenyltetrazolium bromide)) was added. The plates were placed on a shaker for 5 min to mix the formazan and the optical density at 560 nM was measured on a spectrophotometer. Data were plotted using Graphpad prism for calculation of the GI₅₀ concentrations.

Results: All the AML cell lines tested were sensitive to Compound A with GI₅₀ ranging between 2.4 to 7.6 μM. (See Table-1).

TABLE 1
Compound A GI50 (μM) in AML Cell Lines

| Cell Line | U937 | HL60 | THP-1 | KG-1 | MV411 |
|---|---|---|---|---|---|
| GI50 (μM) | 2.4 | 3.5 | 2.5 | 7.6 | 2.5 |

### Example 1A

### Anti-Proliferative Effect of Compound A in the Presence of Uridine Rescue in AML Cell Lines (MTT Assay)

Compound A was tested in the absence of Uridine (U937, HL-60, THP-1 and MV411 cell lines) or in the presence of Uridine (100 μM for U937, HL-60 and MV411 and 300 μM for THP-1). Cells were plated in 96-well plates and incubated with desired concentrations of Compound A for 72 h. At the end of the incubation period, MTT ((3-(4,5-dimethylthiazol-2-yl)-2,5-diphenyltetrazolium bromide)) was added. The plates were placed on a shaker for 5 min to mix the formazan and the optical density at 560 nM was measured on a spectrophotometer. Data were plotted using Graphpad prism for calculation of the GI₅₀ concentrations.

Results: Addition of 100 μM or 300 μM of Uridine caused a rightward shift in the activity of Compound A with GI₅₀>10 μM. (See Table-1A).

TABLE 1A
Compound A GI50 (μM) shift with
Uridine Rescue in AML Cell Lines

| Cell Line | −Uridine | +Uridine |
|---|---|---|
| U937 | 3.2 | >10 |
| THP-1 | 2.0 | >10 |
| HL-60 | 3.6 | >10 |
| MV411 | 2.6 | >10 |

Conclusion. Compound A inhibited growth of AML cell lines with GI₅₀ between 2-3.2 μM and addition of uridine caused rightward shift with GI50>10 μM

### Example 1B

### Anti-Proliferative Effect of Compound A in Combination with Gilteritinib in AML Cell Line THP-1 (MTT Assay)

Compound A (at 3 μM) and Gilteritinib (0.25 μM) was tested in AML cell line THP-1. Cells were plated in 96-well plates and incubated with desired concentrations of Compound A for 72 h. At the end of the incubation period, MTT ((3-(4,5-dimethylthiazol-2-yl)-2,5-diphenyltetrazolium bromide)) was added. The plates were placed on a shaker for 5 min to mix the formazan and the optical density at 560 nM was measured on a spectrophotometer. Data were plotted using Graphpad prism for calculation of the percentage inhibition to determine the effect of Compound A as a single agent or in combination with gilteritinib.

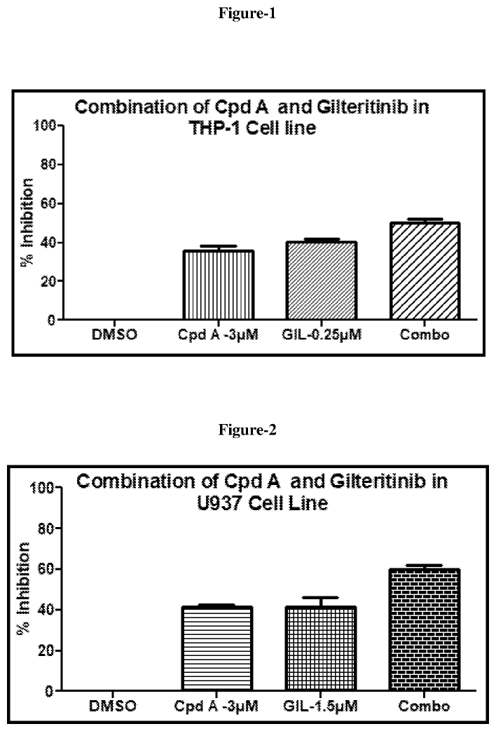

Results: Compound A potentiated (p<0.05) the activity of gilteritinib by inhibiting the cell growth in THP-1 cell lines (See FIG. 1).

TABLE 1B

| Compound | % inhibition |
|---|---|
| Compound A | 35.83 |
| Gilteritinib | 40.10 |
| Compound A + Gilteritinib | 50.20 |

### Example 1C

### Anti-Proliferative Effect of Compound A in Combination with Gilteritinib in AML Cell Line U937 (MTT Assay)

Compound A (at 3 μM) and gilteritinib (1.5 μM) was tested in AML cell line U937. Cells were plated in 96-well plates and incubated with desired concentrations of Compound A for 72 h. At the end of the incubation period, MTT ((3-(4,5-dimethylthiazol-2-yl)-2,5-diphenyltetrazolium bromide)) was added. The plates were placed on a shaker for 5 min to mix the formazan and the optical density at 560 nM was measured on a spectrophotometer. Data were plotted using Graphpad prism for calculation of the percentage inhibition to determine the effect of Compound A as a single agent or in combination with gilteritinib.

Results: Compound A potentiated (p<0.05) the activity of gilteritinib by inhibiting the cell growth in U937 cell lines (See FIG. 2).

TABLE 1C

| Compound | % inhibition |
|---|---|
| Compound A | 41.36 |
| Gilteritinib | 41.66 |
| Compound A + Gilteritinib | 60.13 |

### Example-2

### Effect of Compound A on CD11b mRNA Expression in THP-1 Cell Lines

THP-1 cells were plated in complete media at pre-determined density in 6-well plates and the cells were treated with Compound A for 72 hours. mRNA was isolated using TRI reagent according to the manufacturer's protocol (TRI Reagent from Sigma). cDNA was synthesized using cDNA Synthesis kit according to manufacturer's protocol (First Strand cDNA Synthesis kit) and Real-Time PCR was performed. Data was calculated using delta delta Ct method. Fold change in mRNA expression was plotted using GraphPad Prism (Version 5.02)

Results: Compound A caused differentiation in THP-1 by inducing the CD11b gene expression by 80-fold at 3 μM (See FIG. 3).

### Example-2A

### Effect of Compound A on CD11b Expression in THP-1 and MV411 Cell Lines

Cells were plated in complete media at pre-determined density in 6-well plates and the cells were treated with Compound A 96 hours for THP-1 cell lines and 72 hours for MV411 cell lines) in the presence or absence of uridine. The cells were stained with CD11b Antibody PE according to manufacturer's protocol (CD11b Monoclonal Antibody (ICRF44), PE, eBioscience) and the cells were acquired and analysed by flow cytometry (Guava Easycyte).

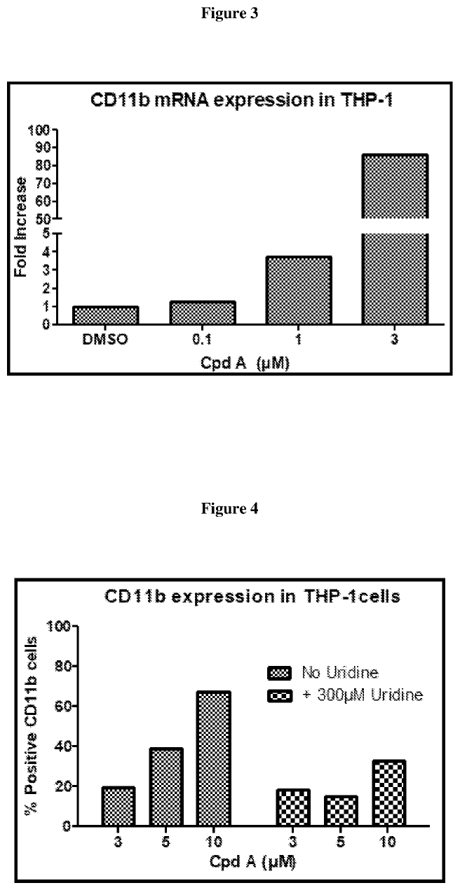

Results: Compound A caused differentiation in THP-1 by inducing the CD11b cell surface expression in 40% of the cell population at 5 μM and addition of uridine reduced the CD11b expression to 15% (See FIG. 4).

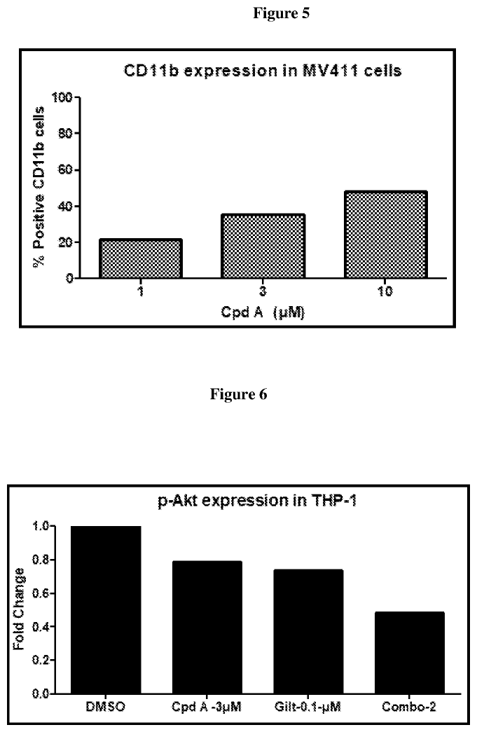

Compound A caused differentiation in MV411 by inducing the CD11b cell surface expression in 35% of the cell population at 3 μM (See FIG. 5).

### Example-3

### Effect of Compound A in Combination with Gilteritinib on p-Akt and p-Erk 1/2 Expressions of AML Cell Line

THP-1 cells were plated in 1% FBS media at pre-determined density in 6-well plates and cells were incubated with Compound A and in combination with gilteritinib for 3 hours. Cells were pelleted, washed with PBS and lysed with lysis buffer (1M Tris-HCl pH 7.5, 1 M NaCl, 0.5 M EDTA pH 8.0, 0.1 M EGTA pH 8.0, protease inhibitor (10×), sodium fluoride, sodium orthovanadate, 200 mM PMSF). Protein estimation was performed using Bradford reagent (ThermoScientific). Samples were denatured, 20 μg of protein was loaded in 7.5% resolving gel for p-Akt and p-Erk 1/2 and SDS Page was performed. Resolved protein was transferred on to the PVDF membrane and probed with anti-rabbit p-Akt and p-Erk 1/2 (1:1000 dilution) primary antibody for overnight at 4° C. Membrane was probed with Ani-rabbit HRP linked IgG secondary antibody at room temperature for 1 hour and ECL substrate was added to the membrane. Membrane was exposed and images were taken in iBright western blot imaging systems. Intensity of the bands were determined using ImageJ 1.42q (NIH, USA) and normalized to β-Actin (loading control). Fold change or percent inhibitions were plotted using GraphPad Prism (Version 5.02).

Results: The combination of Compound A (3 μM) and gilteritinib (0.1 μM) reduced AKT phosphorylation by 54% and p-Erk 1/2 phosphorylation by 58% when compared to gilteritinib alone in the THP-1 cell line (See FIGS. 6 and 7).

### Example-4

### Effect of Compound A in Combination with Cytarabine on MV411 Mouse Xenograft Model

The effect of Compound A was determined in a MV411 mouse xenograft model. Briefly, 5×10⁶ cells were injected into the right flank region by Subcutaneous administration under sterile condition. The oral administration of Compound (A) at 30 mg/kg/BID for 21 days. The tumors were measured using a calliper in two dimensions, length (a) and width (b). Tumor volumes were estimated from measurements of the two diameters of the individual tumors as follows: Tumor Volume (mm3)=(a×b2)/2. At the end of the study period, animals were sacrificed and the tumors harvested.

Results: At the dose tested, Compound A demonstrated significant (P<0.001) anti-tumor activity both as a single agent and in combination with cytarabine at 20 mg/Kg with tumor growth inhibitions of 37 and 73% respectively. No adverse events or body weight changes were observed throughout the study period.

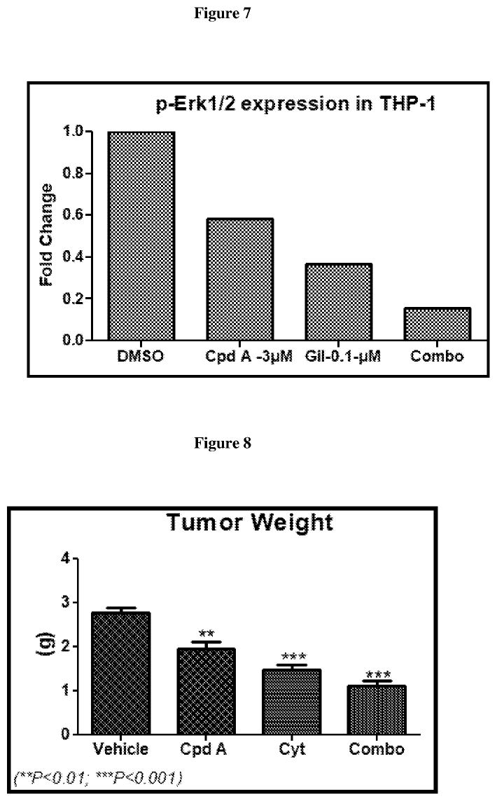

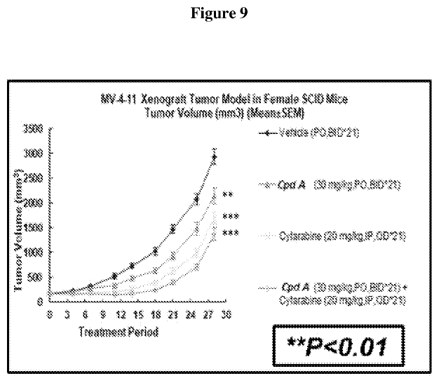

Conclusion: Compound A demonstrated potential in animal models of AML as a single agent or in combination with cytarabine as shown in FIGS. 8 and 9 and the data indicates a therapeutic potential of the Compound A in treatment of AML.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as described above. It is intended that the appended claims define the scope of the invention and that methods and structures within the scope of these claims and their equivalents be covered thereby.

All publications, patents and patent applications cited in this application are herein incorporated by reference to the same extent as if each individual publication, patent or patent application was specifically and individually indicated to be incorporated herein by reference.

1. A method of treating acute myeloid leukemia (AML) comprising administering to a subject in need thereof a dihydroorotate dehydrogenase (DHODH) inhibitor alone or in combination with at least one FLT-3 inhibitor and/or at least one DNA polymerase inhibitor.

2. The method of claim 1, wherein the DHODH inhibitor is selected from leflunomide, teriflunomide, brequinar, dichloroallyl lawsone, maritimus (FK 778), redoxal, DSM265, ASLAN003, PTC299, BRD9185, ML390 and 2-(3′-butoxy-3-chloro-5-fluorobiphenyl-4-ylcarbamoyl)benzoic acid and pharmaceutically acceptable salts thereof and hydrates and solvates thereof.

3. The method of claim 1, wherein the DHODH inhibitor is 2-(3′-butoxy-3-chloro-5-fluorobiphenyl-4-ylcarbamoyl)benzoic acid or a pharmaceutically acceptable salt thereof or a hydrate or solvate thereof.

4. The method of claim 1, wherein the FLT-3 inhibitor is selected from midostaurin, gilteritinib, quizartinib, crenolanib, AKN-028, FF10101, SKLB1028, SKI-G-801, KW-2449, AMG-553, Clifutinib, CHMFL-FLT3-335,N-(4-(6-acetamidopyrimidin-4-yloxy)phenyl)-2-(2-(trifluoromethyl)phenyl)acetamide, SU5614, CG-806, symadex, and pharmaceutically acceptable salts thereof and hydrates and solvates thereof.

5. The method of claim 1 any one of claim 1, wherein the FLT-3 inhibitor is gilteritinib or a pharmaceutically acceptable salt thereof or a hydrate or solvate thereof.

6. The method of claim 1, wherein the DNA polymerase inhibitor is cytarabine or a pharmaceutically acceptable salt thereof or a hydrate or solvate thereof.

7. The method of claim 1, wherein the DHODH inhibitor is administered as a front-line therapy for the acute myeloid leukemia.

8. The method of claim 1, wherein the subject suffers from relapsed-refractory acute myeloid leukemia.

9. The method of claim 1, wherein the subject is a human.

10. The method of claim 1, wherein
(i) the DHODH inhibitor is administered to the subject by the oral, intravenous, intramuscular, or intraperitoneal route;
(ii) the FLT-3 inhibitor is administered to the subject by the oral, intravenous, intramuscular, or intraperitoneal route; and
(iii) the DNA polymerase inhibitor is administered to the subject by the oral, intravenous, intramuscular, or intraperitoneal route.

11. The method of claim 10, wherein
(i) the DHODH inhibitor is administered by the oral route;
(ii) the FLT-3 inhibitor is administered by the oral route; and
(iii) the DNA polymerase inhibitor is administered by the oral route.

12-15. (canceled)

16. The method of claim 1, wherein the DHODH inhibitor is administered at a dose of
about 25 to about 1000 mg.

17-18. (canceled)

19. The method of claim 1, wherein the DHODH inhibitor is administered as a single dose or in divided doses.

20. The method of claim 1, further comprising administering one or more anti-cancer treatments, one or more cytostatic, cytotoxic or anticancer agents, targeted therapy, or any combination of any of the foregoing.

21. The method of claim 20, wherein the the DHODH inhibitor is administered together or sequentially with the one or more anti-cancer treatments, one or more cytostatic, cytotoxic or anticancer agents, targeted therapy, or any combination of any of the foregoing.

22. The method of claim 20, wherein the one or more anticancer agents are selected from DNA interactive agents; topoisomerase II inhibitors; topoisomerase I inhibitors; tubulin interacting agents; hormonal agents; thymidilate synthase inhibitors; and anti-metabolites; other tyrosine kinase inhibitors; angiogenesis inhibitors; EGF inhibitors; VEGF inhibitors; CDK inhibitors; SRC inhibitors; c-Kit inhibitors; Her1/2 inhibitors; monoclonal antibodies directed against growth factor receptors; other protein kinase modulators; CHOP (cyclophosphamide, doxorubicin, vincristine, prednisone); R-CHOP (rituximab-CHOP); hyperCV AD (hyperfractionated cyclophosphamide, vincristine, doxorubicin, dexamethasone, methotrexate, cytarabine); R-hyperCV AD (rituximab-hyperCV AD); FCM (fludarabine, cyclophosphamide, mitoxantrone); R-FCM (rituximab, fludarabine, cyclophosphamide, mitoxantrone); bortezomib and rituximab; temsirolimus and rituximab; temsirolimus and bortezomib; Iodine-131 tositumomab and CHOP; CVP (cyclophosphamide, vincristine, prednisone); R-CVP (rituximab-CVP); ICE (iphosphamide, carboplatin, etoposide); R-ICE (rituximab-ICE); FCR (fludarabine, cyclophosphamide, rituximab); FR (fludarabine, rituximab); and D.T. PACE (Dexamethasone, Thalidomide, Cisplatin, Adriamycin, Cyclophosphamide, Etoposide) and any combination of any of the foregoing.

23. The method of claim 20, wherein the one or more anticancer treatments is selected from chemotherapy, radiation therapy, biological therapy, bone marrow transplantation, stem cell transplant, or any combination of any of the foregoing.

24-42. (canceled)

43. The method of claim 1, wherein the method comprises administering to the subject the DHODH inhibitor (S)-N-(2-chloro-6-fluorophenyl)-4-(4-ethyl-3-(hydroxymethyl)-5-oxo-4,5-dihydro-1H-1,2,4-triazol-1-yl-5-fluoro-2-((1,1,1-trifluoropropan-2-yl)oxy)benzamide or a pharmaceutically acceptable salt thereof in combination with at least one FLT-3 inhibitor and/or a DNA polymerase inhibitor.

44-47. (canceled)

48. A pharmaceutical composition for use in the treatment of acute myeloid leukemia (AML) comprising a dihydroorotate dehydrogenase (DHODH) inhibitor alone or in combination with at least one FLT-3 inhibitor and/or a DNA polymerase inhibitor and a pharmaceutically acceptable carrier.

49. The pharmaceutical composition of claim 48, wherein DHODH inhibitor is leflunomide, teriflunomide, brequinar, dichloroallyl lawsone, maritimus (FK 778), Redoxal, DSM265, ASLAN003, PTC299, BRD9185, ML390 and 2-(3′-butoxy-3-chloro-5 -fluorobiphenyl-4-ylcarbamoyl)benzoic acid or a pharmaceutically acceptable salt thereof.

50. The pharmaceutical composition of claim 48, wherein the DHODH inhibitor is 2-(3′-butoxy-3-chloro-5-fluorobiphenyl-4-ylcarbamoyl)benzoic acid or a pharmaceutically acceptable salt thereof.

51. A pharmaceutical composition for use in the treatment of acute myeloid leukemia (AML) comprising (S)-N-(2-chloro-6-fluorophenyl)-4-(4-ethyl-3(hydroxymethyl)-5-oxo-4,5-dihydro-1H-1,2,4-triazol-1-yl)-5-fluoro-2-((1,1,1-trifluoropropan-2-yl)oxy)benzamide or a pharmaceutically acceptable salt thereof or a hydrate or solvate thereof in combination with at least one FLT-3 inhibitor and/or a DNA polymerase inhibitor and a pharmaceutically acceptable carrier.

52. The pharmaceutical composition of claim 48, wherein the composition further comprises one or more cytostatic, cytotoxic or anticancer agents.
